(19) United States
(12) Patent Application Publication
(10) Pub. No.: US 2014/0081094 A1
(43) Pub. Date: Mar. 20, 2014

(54) METHOD FOR CONSCIOUSNESS AND PAIN MONITORING, MODULE FOR ANALYZING EEG SIGNALS, AND EEG ANESTHESIA MONITOR

(71) Applicants: Denis Jordan, Munich (DE); Eberhard Kochs, Munich (DE)

(72) Inventors: Denis Jordan, Munich (DE); Eberhard Kochs, Munich (DE)

(21) Appl. No.: 14/114,719

(22) PCT Filed: Apr. 26, 2012
(86) PCT No.: PCT/EP2012/001794

§ 371 Date: Oct. 29, 2013

(30) Foreign Application Priority Data
May 2, 2011 (DE) 10 2011 100 173.9
Oct. 6, 2011 (DE) 10 2011 115 116.1

(51) Int. Cl. A61B 5/00; A61B 5/0478; A61B 5/04

(52) U.S. Cl. CPC A61B 5/4821; A61B 5/04017; A61B 5/0478; A61B 5/725; A61B 5/4824
USPC 600/301; 600/544; 600/383

(57) ABSTRACT

A method and apparatus for consciousness and/or pain monitoring, preferably for anesthesia monitoring and for detecting awareness and unconsciousness is described, in which EEG signals are evaluated by means of symbolic transfer entropy. The apparatus includes
a module for analyzing EEG signals, having a data input that can receive and measure EEG signals, a computer unit that can evaluate the EEG signals and an output unit that can output an indicator value for differentiating between awareness and unconsciousness, and to an anesthesia monitor which is configured to measure EEG signals and to evaluate them by means of symbolic transfer entropy.

The invention relates to a method for EEG-based consciousness and pain monitoring, preferably for anesthesia monitoring, to a module for analyzing EEG signals, and to an anesthesia monitor.

Nowadays, general anesthesia is induced by a combination of different anesthetics and is monitored primarily based on nonspecific monitoring parameters (e.g., blood pressure, heart rate, sweating). While these surrogate basic parameters reflect effects of the central action of anesthetics, they do not allow to obtain any direct information about processes in the brain, the main site of action of the hypnotic component of anesthesia. Under these conditions, there is a residual risk of intra-operative awareness, which can lead to a conscious recollection of events during surgery and a severe postoperative neurocognitive stress disorder for the patient.

Conventional patient monitors, also referred to as standard patient monitors below, allow the cardiac rhythm, blood pressure and other non-EEG-based vital parameters, also called basic parameters below, of a patient to be measured and monitored.

To reduce the above-mentioned residual risk, the brain can be monitored more specifically with the aid of a spontaneous electroencephalogram (EEG) than when using the basic parameters. Based on the complex EEG signal, EEG parameters are calculated which are to be used for obtaining a quantification of the hypnotic component of anesthesia (“depth of anesthesia”), in particular a reliable distinction of awareness and unconsciousness. Various mathematical methods are applied as methods of analysis, for example the classical linear spectral analysis. Since the EEG is generated by a nonlinear dynamic system, specific characteristics of the EEG signals may be outside the amplitude spectrum.

The object of the invention is to provide an optimized method for consciousness monitoring (“depth of anesthesia”, hypnotic component of anesthesia, intra-operative awareness, sedation, coma) and/or pain monitoring (analgesia) by an improved evaluation of the nonlinear dynamic properties of the EEG signal, as well as a module for analyzing EEG signals and an EEG anesthesia monitor which allow a correspondingly improved method to be implemented.

This object is achieved by a method for consciousness and pain monitoring, preferably for anesthesia monitoring, in which EEG signals are evaluated by means of symbolic transfer entropy, in particular for differentiating between awareness and unconsciousness. Symbolic transfer entropy allows a quantification of the flow of information between two dynamic systems (referred to as system X and system Y below). Since it is assumed that discontinuing cortical integration in the loss of consciousness is correlated with a change in information transfer at the electrophysiological level, in this way, for example, the hypnotic component can be assessed in anesthesia monitoring. Symbolic transfer entropy is able to specifically quantify this mechanistic process of loss of consciousness. In doing so, instead of analyzing signal amplitudes, only their rank orders are analyzed, and a robust analysis is achieved in this way (low sensitivity to noise and signal interferences). In addition, based on the ordinal approach, the EEG can be analyzed using a small number of data points.

A high reliability of the method can be achieved in that the EEG signals are derived from electrodes, preferably electrode pairs, which are placed at particularly suitable positions. Here it is also possible to evaluate a plurality of electrode pairs.

In particular, intrafrontal, frontal-parietal, frontal-temporal, bitemporal or frontal-occipital electrode leads may be used.

To reduce the influence of undesirable superimposed muscle activity signals (electromyography; EMG) on the EEG signals to be evaluated, that is, to increase the signal-to-noise ratio (SNR) of the measured EEG signals, the EEG signals, preferably prior to a calculation of the symbolic transfer entropy, are low-pass or band-pass filtered with an upper cutoff frequency of 30 Hz at maximum. In the case of band-pass filtering, frequencies within the EEG α-band (8-13 Hz) and/or β-band (13-30 Hz) are particularly suitable.

To avoid aliasing in the sampling of the EEG signals, provision is made for a sampling frequency of the EEG signals which amounts to at least twice, preferably at least five times, the upper filter frequency.

The EEG signals analyzed by the symbolic transfer entropy may be temporal measured value sequences of a duration of 2 to 30 seconds. As a result, even relatively short temporal measured value sequences can be evaluated using the method and short time delays in the range of seconds can be reached for determining the state of consciousness.

For example, subsequences x(i), y(i) of the length m are formed along x, y from N measured values each of the EEG signals within temporal measured value sequences x (measurement of the system X) and y (measurement of the system Y). A lag parameter τ≧1 in the formation of the subsequences may contribute to a better deployment of the trajectories generated from x(i) and y(i) and to a more precise analysis. In the formation of x(i) and y(i), only amplitude values with a time lag τ/fₛ are used here (fₛ sampling frequency of the signals x, y). Symbolic sequences \hat{x}(i), ŷ(i) are then obtained by a symbolization of the subsequences x(i), y(i), preferably by determining the rank order of the amplitudes (ordinal analysis).

It is possible that a directional symbolic transfer entropy is calculated according to the formula

$${STEn}_{Y -> X} = {\sum }_{i} p ( \hat{x} ( i + δ ) , {\hat{x}}_{k} ( i ) , {\hat{y}}_{l} ( i ) ) \cdot log ( \frac{p ( \hat{x} ( i + δ ) \vert {\hat{x}}_{k} ( i ) , {\hat{y}}_{l} ( i ) )}{p ( \hat{x} ( i + δ ) \vert {\hat{x}}_{k} ( i ) )} ) ,$$

where \hat{x}ₖ(i) and ŷₗ(i) each correspond to the k and, respectively, l last symbolic sequences according to the formulas
\hat{x}ₖ(i)=\hat{x}(i), \hat{x}(i−1), . . . , \hat{x}(i−k); ŷₗ(i)=ŷ(i), ŷ(i−1), . . . , ŷ(i−l) and the sum across all sequences \hat{x}(i+δ), \hat{x}(i), ŷₗ(i) is formed. This expresses the probability of the extrinsic predictability of a sequence \hat{x}(i+δ) with δ>0 from information in ŷₗ(i). The directional symbolic transfer entropy is thus a measure of the extent to which subsequences from a measured value sequence can be explained by preceding subsequences from the other measured value sequence. By an interchange of the respective subsequences of the systems X and Y, the directional symbolic transfer entropy STEn_{X→Y}, can be calculated accordingly.

The time offset δ is preferably selected such that the quotient of sampling frequency and time offset is within the frequency range of the EEG α-, β-band.

It is additionally possible that a direction index STEn_{DI} is calculated according to the formula STEn_{DI}=STEn_{X→Y}−STEn_{Y→X}. When communication exists, a value of 0 (STEn_{Y→X}+STEn_{X→Y}>0) represents a balanced bidirectional exchange of information between X and Y; for positive values, the system X is predominantly the generator, for negative values, the system Y is predominantly the generator.

To allow a simple and quick assessment to be made of the results of the analysis of the EEG signals, for example by a physician in anesthesia monitoring, an indicator value for indicating the state of consciousness and/or of pain is established, primarily for differentiating between awareness and unconsciousness based on the evaluation of the EEG signals by means of symbolic transfer entropy and possible further parameters of the EEG and/or of the basic monitoring (cardiovascular system, respiration as well as patient data and drug information). The combination of symbolic transfer entropy with further parameters to form an indicator may be effected with the aid of a fuzzy logic, neural networks, support vector machines or regressions. The indicator may be used for monitoring or automatically controlling the hypnotic and/or analgesic component of anesthesia.

The object of the invention is further achieved by a module for analyzing EEG signals, including a data input that can receive EEG signals, a computer unit that can evaluate the EEG signals in accordance with a method according to any of the preceding claims, and an output unit that can output an indicator value for determining the state of consciousness or of pain, preferably for differentiating between awareness and unconsciousness. This allows a modular design of a system for analyzing EEG signals, the module being, for example, adapted to receive EEG signals from a separate EEG amplifier and outputting the indicator value to a conventional patient monitor.

It is also possible for the module to include an EEG amplifier which is firmly integrated in the module or forms a separate, portable, preferably wireless unit and provides EEG signals to the data input of the module.

The module may further include an interface for a conventional patient monitor, the interface being adapted to receive non-EEG parameters, and the computer unit being configured to be adapted to determine the indicator value taking into account the non-EEG parameters.

An EEG anesthesia monitor according to the invention is configured to measure EEG signals and to evaluate them by means of symbolic transfer entropy, preferably in accordance with a method of claims 1 to 11, in particular to allow to differentiate between awareness and unconsciousness.

A module as described above and an independent EEG anesthesia monitor may also be used in other fields of application in addition to anesthesia monitoring or control, in particular in patient monitoring in intensive care units, for example in the case of sedation or coma, for sleep monitoring in sleep research or for vigilance monitoring of participants in traffic, for example pilots, truck drivers or bus drivers.

Further features and advantages of the invention will be apparent from the description below and from the drawings, to which reference is made and in which:

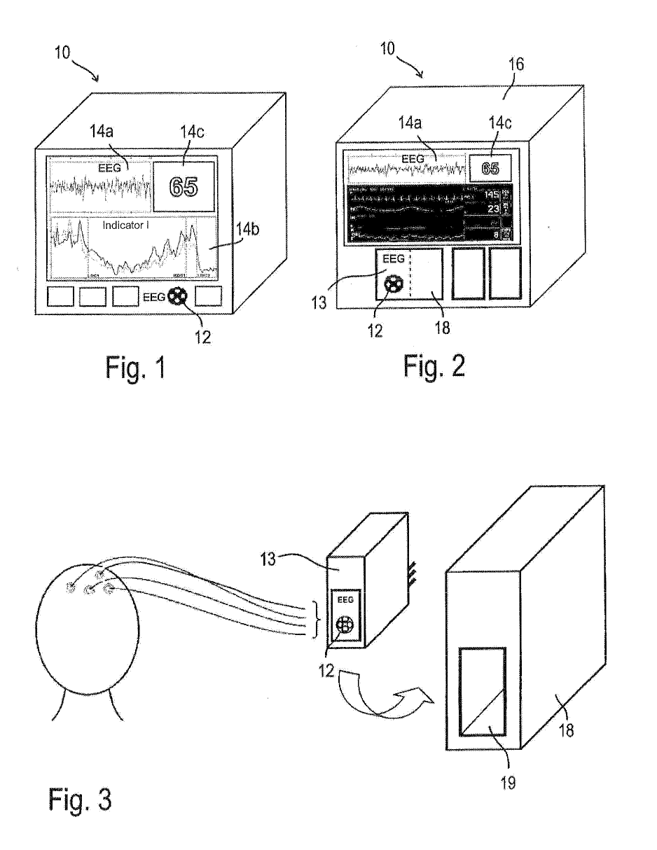

FIG. 1 shows an EEG anesthesia monitor according to the invention;

FIG. 2 shows an anesthesia monitor with a module according to the invention for analyzing EEG signals;

FIG. 3 shows a module according to the invention for analyzing EEG signals;

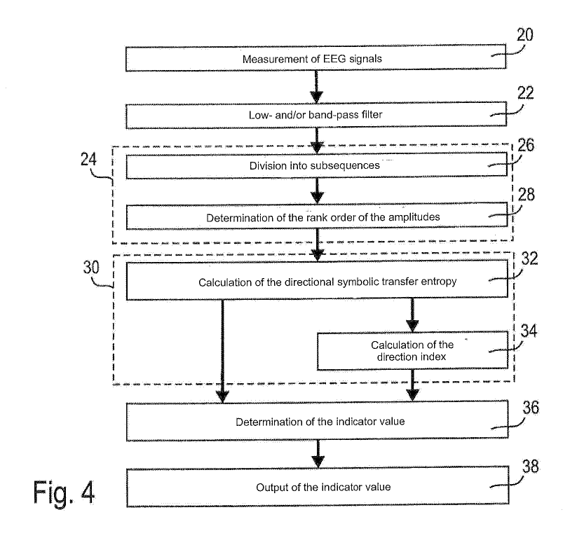

FIG. 4 shows a flow chart of a method according to the invention for consciousness monitoring;

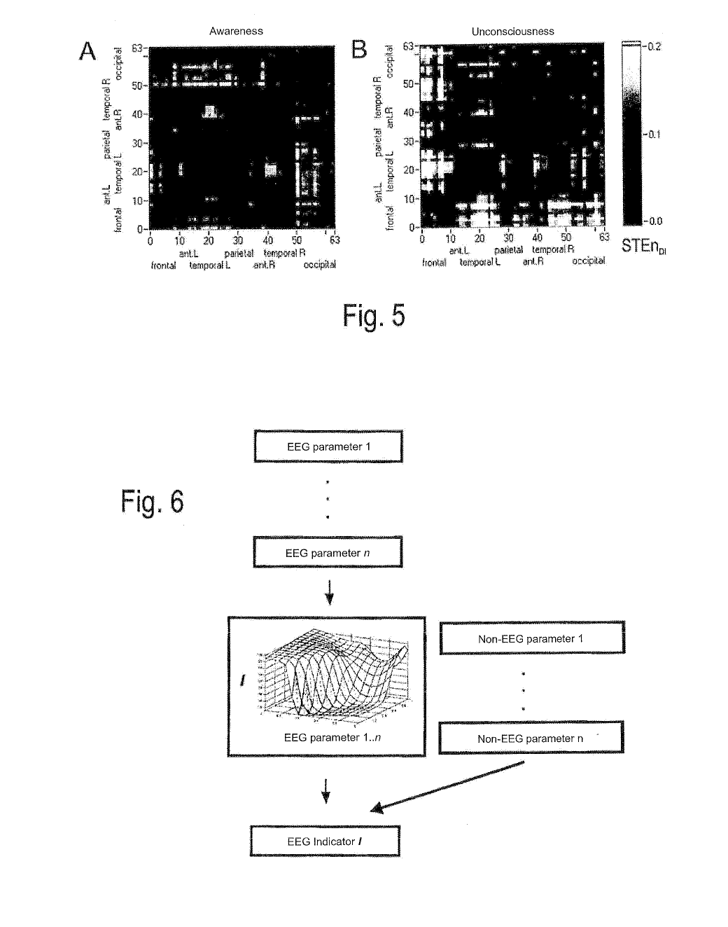

FIG. 5 shows a graphical representation of the symbolic transfer entropy of EEG signals (64 channels) for relaxed awareness and unconsciousness; and

FIG. 6 shows a flow chart of the determination of an indicator value from individual EEG parameters and optional non-EEG parameters in accordance with a method according to the invention.

FIG. 1 shows an EEG anesthesia monitor 10 by which EEG signals can be evaluated by means of symbolic transfer entropy. The anesthesia monitor 10 has a connection 12 for a plurality of EEG electrodes which are arranged on the scalp of a patient and serve to record the EEG. The anesthesia monitor 10 evaluates, by means of symbolic transfer entropy, the EEG signals received from the electrodes, an indicator value I being determined, especially for differentiating between awareness and unconsciousness.

The anesthesia monitor 10 includes a first display 14a which displays the EEG signals, a second display 14b which displays the indicator value I over time, and a third display 14c which displays the current indicator value I. This allows a physician to make a simple and quick assessment of the depth of anesthesia during anesthesia monitoring.

The anesthesia monitor 10 according to FIG. 1 is designed as an independent apparatus; in addition to the determination and display of the indicator value I as the result of the EEG signal analysis by means of symbolic transfer entropy and possible additional EEG parameters and basic parameters, further functions for evaluating the EEG signals and/or for assessing the state of consciousness and/or pain may also be provided.

FIG. 2 shows an alternative embodiment of an anesthesia monitor 10 having a conventional, known standard patient monitor 16 for anesthesia which is equipped with an additional module 18 allowing an evaluation of the EEG signals by means of symbolic transfer entropy.

Apart from connections for the power supply by the standard patient monitor 16, the module 18 includes a data input with an integrated EEG amplifier 13 which can directly measure or receive EEG signals, a computer unit which can evaluate the EEG signals by means of symbolic transfer entropy and possible further EEG and basic parameters, and an interface with the standard patient monitor 16 by which the calculated indicator based on symbolic transfer entropy with a possible combination of further EEG parameters with/without taking basic parameters into account and the measured EEG is represented on the output unit. In the embodiment shown, the indicator value I is transmitted to the standard patient monitor 16 and displayed on the display 14c thereof.

FIG. 3 shows a variant of a module 18, which, in contrast to the integrated EEG amplifier having the socket 12, includes a mobile EEG amplifier 13. The EEG amplifier 13 is configured as a separate, portable unit including an accumulator for energy supply and allows a wireless transfer of the EEG signals to the data input of the module 18. The EEG amplifier may thus be placed near the patient without restricting the location of the monitor.

The module 18 includes a slot 19 which can be used for inserting the EEG amplifier 13. In this way, the accumulator can be charged and/or the EEG amplifier 13 can be supplied with energy via the module 18.

It is also possible that the module 18 is designed without an EEG amplifier and receives EEG signals from a separate external EEG amplifier via its data input.

A modular design of this type allows the use of known components, such as conventional patient monitors, with a module 18 for deriving and analyzing EEG signals by means of symbolic transfer entropy. The module 18 may also be configured to perform selected functions or all functions of these components.

In addition to the application in an EEG anesthesia monitor 10 or as a module 18 in conjunction with a standard patient monitor 16, symbolic transfer entropy and the calculated indicator I and the module 18 may also be used in further fields of application, which may include, more particularly, patient monitoring in intensive care units, in particular in the case of sedation or coma, sleep monitoring, and vigilance monitoring of participants in traffic, for example pilots or truck or bus drivers.

Depending on the field of application, the module 18 can be used with components of different configurations, such as, for example, conventional patient monitors.

More particularly, it is also possible that only one electrode pair is provided for the module 18 and for the EEG monitor.

A method for consciousness and/or pain monitoring, in particular for anesthesia monitoring, in an EEG anesthesia monitor 10 or in a module 13 with a standard patient monitor 16 of FIG. 1 or 2 will now be described below with reference to FIGS. 4, 5 and 6.

In a first step 20, the EEG signals are measured. Suitable for this are, above all, intrafrontal (e.g., Fp1-Fp2 in the internationally standardized 10-20 system), frontal-parietal (e.g., Fpz-Pz), frontal-temporal (e.g., Fp2-FT9), bitemporal (e.g., FT9-FT10), and frontal-occipital (e.g., Fpz-Oz) electrode leads. Preferably, two of these pairs are used.

In a subsequent step 22, the EEG signals are low-pass filtered with a cutoff frequency of 30 Hz at the maximum. As an alternative, the EEG signals may be band-pass filtered. In the case of a band-pass filtering, frequencies within the EEG α-band (8-13 Hz) and/or β-band (13-30 Hz) are particularly suitable. In this way, the influence of muscle activity in the EEG is reduced, such muscle activity leading to a poor SNR of the EEG, particularly in high frequencies of the EEG γ-band above 30 Hz.

The EEG signals analyzed by symbolic transfer entropy are temporal measured value sequences of a duration of 2 to 30 seconds, which are determined at a predefined sampling frequency fₛ. In the variant of the method as described, the time duration of the measured value sequences is 10 seconds and the sampling frequency fₛ is 200 Hz. The temporal measured value sequence thus comprises 2000 measuring points.

The sampling frequency fₛ of the EEG signals and the upper filter frequency of the low-pass filter or of the band-pass filter are adjusted to each other such that the sampling frequency fₛ of the EEG signals amounts to at least twice the upper filter frequency. In this way, aliasing is avoided.

After filtering the measured value sequences, a symbolization 24 is effected. In the variant shown, a division 26 of the temporal measured value sequences x, y of an electrode pair with N measured values each into subsequences x(i), y(i) of the length m is performed. In this way, in each case up to N−m+1(τ=1) subsequences x(i) and, respectively, y(i) are obtained, which are reduced in the case of lag τ>1. In the present case, τ=1 is used; in the case of higher values the trajectories formed from the subsequences are deployed in the m-dimensional Euclidean space, whereby a more accurate analysis is possibly reached by the symbolic transfer entropy. When fₛ=200 Hz, values from 1 to 10 are particularly suitable. The length m of the subsequences amounts to at least 3, but should meet m!≦N for a correct calculation; in the embodiment described, the length of the subsequences is equal to 3. In this way, good results can be achieved involving comparatively little computing expenditure.

In a following method step 28, these subsequences are symbolized by determining the rank order of the amplitudes (ordinal analysis), as a result of which symbolic sequences \hat{x}(i) and, respectively, ŷ(i) are obtained.

The symbolic sequences \hat{x}(i) and ŷ(i) are used for a calculation 30 of the symbolic transfer entropy. Various entropy measures are subsumed under the term of symbolic transfer entropy here.

In a first step 32, a directional symbolic transfer entropy STEn_{y→x} is calculated according to the formula

$${STEn}_{Y -> X} = {\sum }_{i} p ( \hat{x} ( i + δ ) , {\hat{x}}_{k} ( i ) , {\hat{y}}_{l} ( i ) ) \cdot log ( \frac{p ( \hat{x} ( i + δ ) \vert {\hat{x}}_{k} ( i ) , {\hat{y}}_{l} ( i ) )}{p ( \hat{x} ( i + δ ) \vert {\hat{x}}_{k} ( i ) )} ) .$$

\hat{x}ₖ(i) and ŷₗ(i) each correspond to the k and, respectively, l last symbolic sequences according to the formulas

\hat{x}ₖ(i)=\hat{x}(i),\hat{x}(i−1), . . . ,\hat{x}(i−k);ŷₗ(i)=ŷ(i),ŷ(i−1), . . . ,ŷ(i−l).

In the present variant of the method, the depth of predictability is limited to a sequence preceding \hat{x}(i+δ) at a distance δ, that is, k and l are set equal to zero. But it is also possible that k and l may be selected greater than zero.

The directional symbolic transfer entropy is derived from the Shannon entropy and a conditional Kullback-Leibler entropy.

The common probability that the symbolic sequence \hat{x}(i+δ) appears with the preceding symbolic sequences \hat{x}ₖ(i) and ŷₗ(i) is p(\hat{x}(i+δ), \hat{x}ₖ(i), ŷₗ(i)).

The conditional probability that the symbolic sequence (5) occurs under the condition of the preceding symbolic sequences \hat{x}ₖ(i) and ŷₗ(i) is p(\hat{x}(i+δ)|\hat{x}ₖ(i), ŷₗ(i)).

The conditional probability that the symbolic sequence \hat{x}(i+δ) occurs under the condition of the preceding symbolic sequence \hat{x}ₖ(i) is given by p(\hat{x}(i+δ)|\hat{x}ₖ(i)).

Analogously, a directional symbolic transfer entropy STEn_{X→Y} can be calculated, the respective subsequences of the two systems X and Y being interchanged.

A time offset δ is indicated by a number of measuring points of the temporal measured value sequences. The actual temporal offset thus results from the time offset δ and the sampling frequency fₛ.

In the method variant presented here, the sampling frequency fₛ is 200 Hz and the time offset δ corresponds to 10 measured values. In this way, the quotient of the sampling frequency fₛ and the time offset 6, being 20 Hz, is within the frequency range of the EEG β-band. Taking into consideration the previously mentioned conditions for the sampling frequency, δ and fₛ may essentially be varied as desired, as long as their quotient is within the frequency range of the EEG analyzed, preferably in the α- or β-band.

A further measure of the symbolic transfer entropy is constituted by the direction index STEn_{DI}, which is calculated in a further method step 34 by the difference of the two associated directional symbolic transfer entropies:

STEn_{DI}=STEn_{X→Y}−STEn_{Y→X}

The direction index STEn_{DI} defines and determines the preferred direction of the information flow between the two systems. When a communication exists, a value of 0 represents a balanced bidirectional exchange of information between X and Y. For positive values, the system X predominantly is the generator.

FIG. 5 illustrates a graphic representation of the direction index STEn_{DI} for relaxed awareness in the image area A and for unconsciousness in the image area B. For the sake of simplicity, the absolute value of the direction index STEn_{DI} is plotted, with lower values of the direction index STEn_{DI} being shown dark and higher values light. The graphic representation shows the results of the symbolic transfer entropy, which was calculated with the aid of 64-channel EEG data in 15 volunteers in a state of relaxed awareness and propofol-induced unconsciousness, and effects of propofol above all in electrode combinations taking a frontal electrode into account.

While in the state of relaxed awareness, for the most part a balanced flow of information between the electrode pairs is observed in the image area A with corresponding low values of the direction index STEn_{DI}, an unbalanced exchange of information is predominant during unconsciousness in image B, characterized by the lighter coloration and correspondingly higher values of the direction index STEn_{DI}. This is observed in particular in frontal-temporal, frontal-parietal and occipital electrode combinations. In terms of quality, this result is in line with imaging and high spatial resolution fMRI examinations during anesthesia, which are indicative of a suppression of the cortico-cortical connectivity of the network architecture, in particular default and higher executive networks.

The calculation 30 of the symbolic transfer entropy is followed by the determination 36 of an indicator value I, as illustrated in FIG. 6. Here, a plurality of EEG parameters and/or non-EEG parameters of basic monitoring (cardiovascular system, respiration as well as patient data and drug information), 1 to n, is evaluated and an individual indicator value I is determined. The parameters 1 to n more particularly comprise the symbolic transfer entropy measures STEn_{DI}, STEn_{X→Y} and STEn_{Y→X} determined in the preceding method steps 32, 34.

The indicator value I is, for example, defined such that it can assume values between 0 and 100, with values between 80 and 100 corresponding to awareness and values between 0 and 20 corresponding to a deep anesthesia. In addition to the above-mentioned parameters of symbolic transfer entropy, further EEG parameters or further non-EEG parameters (basic monitoring parameters including patient data and drug information) may also contribute to determining the indicator value I.

In a final method step 38, the indicator value I is output, the indicator value either being displayed as an independent output value or entering into the determination of a further indicator value in an anesthesia monitor together with other parameters.

The method described above for consciousness monitoring is suitable for both sexes and all age groups. However, depending on the sex or age or according to the field of application, different parameters may be used, it being possible to vary the parameters, starting with the arrangement and number of the electrodes up to the parameters in calculating the directional symbolic transfer entropy, for example of the time offset δ. In addition, the method may be employed for different combinations of anesthetics having a hypnotic and analgesic effect and may be configured specially for specific drug protocols.

The approach of symbolic transfer entropy is close to the underlying neuronal processes. To this end, the cortico-cortical coupling can be detected and quantified on the informational level by time series of the electrical potentials of specific electrodes. The symbolic transfer entropy here specifically addresses mechanistic effects of a drug-induced loss of consciousness. This approach is advantageous because unconsciousness is directly correlated with impaired information processing. The preliminary examinations carried out based on the high-resolution EEG data in volunteers under propofol anesthesia show that the symbolic transfer entropy, as a new EEG parameter for anesthesia monitoring, achieves a particularly good differentiation between awareness and unconsciousness, exceeding the current state of the art.

Symbolic transfer entropy can also be employed for adjacent applications in connection with sedation, sleep and coma monitoring.

1. A method for consciousness and/or pain monitoring, preferably for anesthesia monitoring, in which EEG signals are evaluated by means of symbolic transfer entropy, in particular for differentiating between awareness and unconsciousness.

2. The method according to claim 1, wherein the EEG signals from a plurality of electrodes, preferably one or a plurality of electrode pairs, are evaluated.

3. The method according to claim 1, wherein intrafrontal, frontal-parietal, frontal-temporal, bitemporal or frontal-occipital electrode leads are provided.

4. The method according to claim 1, wherein prior to a calculation of the symbolic transfer entropy, the EEG signals are low-pass filtered and/or band-pass filtered with a cutoff frequency of 30 Hz at maximum, the bandwidth preferably being within the frequency band of 8 Hz to 30 Hz.

5. The method according to claim 4, wherein a sampling frequency of the EEG signals is provided which amounts to at least twice, preferably at least five times the upper filter frequency.

6. The method according to claim 1, wherein EEG signals are temporal measured value sequences of a duration of 2 to 30 seconds.

7. The method according to claim 1, wherein from temporal measured value sequences x, y with N measured values of the EEG signals, subsequences x(i), y(i) of a length m with lag i are formed along x and y and symbolic sequences \hat{x}(i), ŷ(i) are obtained by a symbolization of the subsequences x(i), y(i), preferably by determining the rank order of the amplitudes, where m is equal to or greater than 3, preferably exactly 3.

8. The method according to claim 1, wherein a directional symbolic transfer entropy is calculated according to the formula
$${STEn}_{Y -> X} = {\sum }_{i} p ( \hat{x} ( i + δ ) , {\hat{x}}_{k} ( i ) , {\hat{y}}_{l} ( i ) ) \cdot log ( \frac{p ( \hat{x} ( i + δ ) \vert {\hat{x}}_{k} ( i ) , {\hat{y}}_{l} ( i ) )}{p ( \hat{x} ( i + δ ) \vert {\hat{x}}_{k} ( i ) )} )$$
where k and l for
\hat{x}ₖ(i)=\hat{x}(i),\hat{x}(i−1), . . . ,\hat{x}(i−k);ŷₗ(i)=ŷ(i),ŷ(i−1), . . . ,ŷ(i−l).
are preferably zero.

9. The method according to claim 8, wherein the time offset δ is selected such that the quotient of sampling frequency and time offset is within the frequency range of the EEG α- or β-band.

10. The method according to claim 8, wherein a direction index STEn_{DI} is calculated according to the formula
STEn_{DI}=STEn_{X→Y}−STEn_{Y→X}.

11. The method according to claim 1, wherein an indicator value is established for differentiating between awareness and unconsciousness based on the evaluation of the EEG signals by symbolic transfer entropy.

12. A module for analyzing EEG signals, comprising a data input that can receive EEG signals, a computer unit that can evaluate the EEG signals in accordance with the method of claim 1, and an output unit that can output an indicator value for differentiating between awareness and unconsciousness.

13. The module according to claim 12, wherein an EEG amplifier is provided which is firmly integrated in the module or forms a separate, portable, preferably wireless unit and provides EEG signals to the data input of the module.

14. The module according to claim 12, wherein, module includes an interface for a conventional patient monitor, the interface being adapted to receive non-EEG parameters, the computer unit being configured to be adapted to determine the indicator value taking into account the non-EEG parameters.

15. An EEG anesthesia monitor which is configured to measure EEG signals and to evaluate them by symbolic transfer entropy in accordance with the method of claim 1, in particular to allow to differentiate between awareness and unconsciousness.

16. The method according to claim 2, wherein that intrafrontal, frontal-parietal, frontal-temporal, bitemporal or frontal-occipital electrode leads are provided.

17. The module according to claim 13, wherein the module includes an interface for a conventional patient monitor, the interface being adapted to receive non-EEG parameters, the computer unit being configured to be adapted to determine the indicator value taking into account the non-EEG parameters.
